(19) United States
(12) Patent Application Publication
(10) Pub. No.: US 2022/0192632 A1
(43) Pub. Date: Jun. 23, 2022

(54) METHOD AND APPARATUS FOR BREAST IMAGING

(71) Applicant: Shabbir Bakir Bambot, West Hills, CA (US)

(72) Inventor: Shabbir Bakir Bambot, West Hills, CA (US)

(21) Appl. No.: 17/446,079

(22) Filed: Aug. 26, 2021

Related U.S. Application Data
(60) Provisional application No. 63/194,455, filed on May 28, 2021.
(60) Provisional application No. 63/128,173, filed on Dec. 20, 2020.

(51) Int. Cl. A61B 8/00; A61B 8/08

(52) U.S. Cl. CPC A61B 8/403; A61B 8/4281; A61B 8/4416; A61B 8/0825

(57) ABSTRACT

An imaging system is provided that includes a compression and scanning assembly including a frame that receives a compression element configured to compress breast tissue. The compression element includes an open region configured to receive an ultrasound transducer, and the open region is configured to move relative to the breast tissue after the compression and scanning assembly has been secured to the breast tissue.

### RELATED APPLICATION

The present application claims priority to U.S. Provisional Application No. 63/194,455, Titled “METHOD AND APPARATUS FOR AUTOMATED BREAST ULTRASOUND” which was filed on 28 May 2021, and U.S. Provisional Application No. 63/128,173, Titled “METHOD AND APPARATUS FOR AUTOMATED BREAST ULTRASOUND” which was filed 20 Dec. 2020 the complete subject matter of each which is expressly incorporated herein by reference in their entireties.

### BACKGROUND

The present application relates generally to the field of cancer imaging and diagnosis and, more specifically, to breast cancer imaging either as a standalone procedure or in combination with mammography or tomosynthesis for acquiring breast tissue images for the purpose of detecting cancer.

Breast cancer is the second most common cancer among women overall and is the most common cause of cancer related death among minority populations in the US. Despite its success in reducing breast cancer mortality, X-ray mammography continues to miss cancer in a significant number of women and particularly in women with dense breast tissue. Supplemental screening with ultrasound has become an important tool in helping find early cancer in these women as it provides information that is complementary to mammography. The primary cues indicative of breast cancer are microcalcifications, architectural distortions and masses. Mammographic findings that suggest a breast cancer may be present are microcalcifications, architectural distortion, and masses. Ultrasound is most useful for further characterization of masses, allowing for distinction between potentially suspicious solid masses and benign cysts. Ultrasound is also an important alternate tool and is routinely used to target breast tissue biopsy.

During a diagnostic ultrasound procedure, good contact between the transducer and tissue is necessary and the presence of air in the path of sound waves is undesirable. Air acts as a sound barrier that degrades image resolution and sound waves passing through the body are blocked by air pockets, however small, if they are present during an ultrasound scanning procedure. Therefore, eliminating air between the transducer and the patient's skin improves imaging quality. Meanwhile, for mammography, vigorous breast compression is necessary to help spread out the breast tissue for better visualization and to reduce thickness. The reduced thickness helps reduce x-ray dose.

While manual ultrasound scanning provides good image quality and accuracy, manual scanning is highly dependent upon the ability and knowledge of the clinician in manipulating the ultrasound transducer. Such manipulation includes applying pressure, tilting, twisting, and rocking the transducer to change the angle of insertion of ultrasound energy into the tissue in order to improve the visibility of features in the body and to eliminate image artifacts.

In addition, acquiring mammography/tomosynthesis and ultrasound imaging data at roughly the same time and with the patient in the same position has also proven problematic. In particular, there is an inability to address the somewhat opposing needs of the two modalities. Mammography requires vigorous compression of the breast, and this is done using a rigid compression paddle. To image the breast in the same configuration as mammography, attempts have therefore been made to scan the breast through the material of the rigid compression paddle with an ultrasound transducer or probe whilst maintaining as near the same compression and patient position as mammography.

Further, most ultrasound breast tissue exams are done manually by experienced clinicians using handheld transducers. The patient is examined while sitting or supine with an ultrasound transducer manipulated to scan over the breast tissue region requiring further examination. Manual ultrasound scanning provides the best image quality and highest accuracy. However, this is highly dependent upon the ability and knowledge of the clinician in seeing and recognizing internal tissue features and his or her experience and skill in handling and manipulating the ultrasound transducer on the breast tissue surface to better visualize these features. Such manipulation includes applying pressure, tilting, twisting, and rocking the transducer to change the angle of insertion of ultrasound energy into the tissue in order to improve the visibility of features in the body and to eliminate image artifacts. However, great variability in experience and skill among clinicians leads to variability and a lack of standardization in the accuracy of manual breast tissue ultrasound. The ability to perform manual examinations is also limited by shortages in skilled clinicians.

Automated breast ultrasound systems (ABUS) have been introduced recently to help standardize and make breast tissue ultrasound exams operator independent. Examples include ABUS systems such as the GE Invenia™ or prone as in the Hitachi Sofia™ 3D breast ultrasound system. The breast tissue of the patient, while lying either supine or prone, is automatically scanned with an ultrasound transducer in a system that is pressed against the breast tissue. In these systems an ultrasound transducer is made to scan the breast tissue whilst being compressed by a taut mesh material, fabric or membranous sheet as taught in U.S. Pat. Nos. 6,932,768, 9,949,719 and 10,561,394, all of which are incorporated herein as a reference. Still, difficulties in efficiently transferring and coupling the ultrasound energy into the tissue remain.

Furthermore, the manner in which most mammography and sonography (ultrasound imaging) is performed today limits the potential for a better, more accurate, cost-effective, and timely diagnosis. This is because the images in each mode are acquired with the patient in different positions, upright for the mammogram and sitting, supine or prone for ultrasound. As a result, co-registration of abnormalities seen in any one modality with the other becomes a challenge especially when multi-focal disease is present and is further aggravated by the deformable nature of the breast tissue. Moreover, mammograms and sonograms require separate equipment often located in separate exam rooms and consequently may not be acquired on the same day or the same facility leading to a time delay between procedures and multiple hand-offs that can result in the loss of critical information.

Further, there remains a strong unresolved and continuing need for a system and method for fast and efficient ultrasound imaging of the entire breast tissue in the same patient configuration as mammography/tomosynthesis. It is desirable to conduct both mammography and ultrasound in roughly the same time or the same patient encounter and preferably using the same equipment.

### SUMMARY

In accordance with embodiments herein, an imaging system is provided that includes a compression and scanning assembly including a frame that receives a compression element configured to compress breast tissue. The compression element includes an open region configured to receive an ultrasound transducer, and the open region is configured to move relative to the breast tissue after the compression and scanning assembly has been secured to the breast tissue.

Optionally, the imaging system also includes an articulating arm coupled to the compression and scanning assembly and configured to permit movement of the compression and scanning assembly. In one aspect, the imaging system additionally includes a work station coupled to the articulating arm, and including one or more processors configured to move the articulating arm, and operate the ultrasound transducer to move the ultrasound transducer relative to the compression and scanning assembly. In another aspect, the compression element is a polymer membrane or a polymer mesh material. In an example, the compression element includes at least one movable element configured to actuate to move the open region from a first position to a second position, In yet another example, the compression element is a compression paddle that includes a first movable element spaced from a second movable element to form a channel therebetween. Optionally, the first movable element is removable from the compression paddle. Alternatively, the first movable element is hingedly coupled to the second moveable element.

Optionally, the compression element includes at least one movable element that is a roller element. In one aspect, the imaging system also includes an x-ray detector coupled to the compression and scanning assembly. In another aspect, the imaging system additionally includes a movable arm configured to receive the ultrasound transducer and to move the ultrasound transducer away from the x-ray detector or an x-ray imaging area. In one example, the compression and scanning assembly further comprises a rock and tilt assembly received within the frame and configured to receive the ultrasound transducer. In another example, the rock and tilt assembly includes a subframe that is configured to receive the ultrasound transducer and is slidably coupled to the frame with a lower pin element and an upper pin element, wherein the subframe is configured to rotate the ultrasound transducer about the lower pin element and upper pin element.

In one or more embodiments, a method for imaging is provided that includes compressing breast tissue to a first thickness with a compression element of a compression and scanning assembly, x-ray imaging the breast tissue at the first thickness with the compression and scanning assembly to obtain an x-ray image, disposing an ultrasound traducer through an open region in the compression element to directly compress the breast tissue, and moving the ultrasound transducer across the breast tissue as the open region is modified in the compression element to obtain an ultrasound image.

Optionally, the method also includes applying gel to the breast tissue through channels in the compression element of the compression and scanning assembly prior to disposing the ultrasound transducer through the open region in the compression element. In one aspect, the x-ray imaging the breast tissue at the first thickness with the compression and scanning assembly to obtain an x-ray image includes reconstructing an initial x-ray image to form the x-ray image. In another aspect, the method includes modifying compression of the breast tissue with the compression and scanning assembly after x-ray imaging the breast tissue and before disposing the ultrasound transducer through the open region of the compression element. In one example, modifying the compression of the breast tissue includes obtaining feedback related to a compression force measured by at least one sensor, and modifying the compression based on the feedback obtained.

In one or more embodiments, an imaging system is provided that includes a compression and scanning assembly including a frame that receives a compression element configured to compress breast tissue. The compression element includes moving elements with an open region formed within the moving element, the moving elements configured to receive an ultrasound transducer within the open region. A position of the open region in the moving elements is configured to move relative to the breast tissue after the compression and scanning assembly has been secured to the breast tissue. The imaging system also includes an x-ray detector coupled to the compression and scanning assembly for obtaining x-ray images. Optionally, the imaging system also includes a support arm coupled to the compression and scanning assembly and configured to move the compression and scanning assembly away from the x-ray imaging area when obtaining the x-ray mammography images.

### BRIEF DESCRIPTION OF THE DRAWINGS

FIG. 1 illustrates a schematic view of an imaging system in accordance with one embodiment of the present disclosure.

FIG. 2 is a perspective view of a compression and scanning assembly in accordance with one embodiment of the present disclosure.

FIG. 3 is a plan view of a compression and scanning assembly in accordance with one embodiment of the present disclosure.

FIG. 4 is an axial sectional view of a compression and scanning assembly in accordance with one embodiment of the present disclosure.

FIG. 5A is a top plan view of an automated breast ultrasound (ABUS) in accordance with one embodiment of the present disclosure.

FIG. 5B is a top plan view of an automated breast ultrasound (ABUS) in accordance with one embodiment of the present disclosure.

FIG. 6 is a perspective view of a compression element in accordance with one embodiment of the present disclosure.

FIG. 7A is a perspective view of a compression element in accordance with one embodiment of the present disclosure.

FIG. 7B is a perspective view of a compression element in accordance with one embodiment of the present disclosure.

FIG. 7C is a perspective view of a compression element in accordance with one embodiment of the present disclosure.

FIG. 7D is a perspective view of a compression element in accordance with one embodiment of the present disclosure.

FIG. 7E is a perspective view of a compression element in accordance with one embodiment of the present disclosure.

FIG. 7F is a perspective view of a compression element in accordance with one embodiment of the present disclosure.

FIG. 7G is a perspective view of a compression element in accordance with one embodiment of the present disclosure.

FIG. 7H is a perspective view of a compression element in accordance with one embodiment of the present disclosure.

FIG. 8 is a perspective view of an imaging system in accordance with one embodiment of the present disclosure.

FIG. 9 is a perspective view of an imaging system in accordance with one embodiment of the present disclosure.

FIG. 10 is a schematic block flow diagram of a process for imaging in accordance with one embodiment of the present disclosure.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

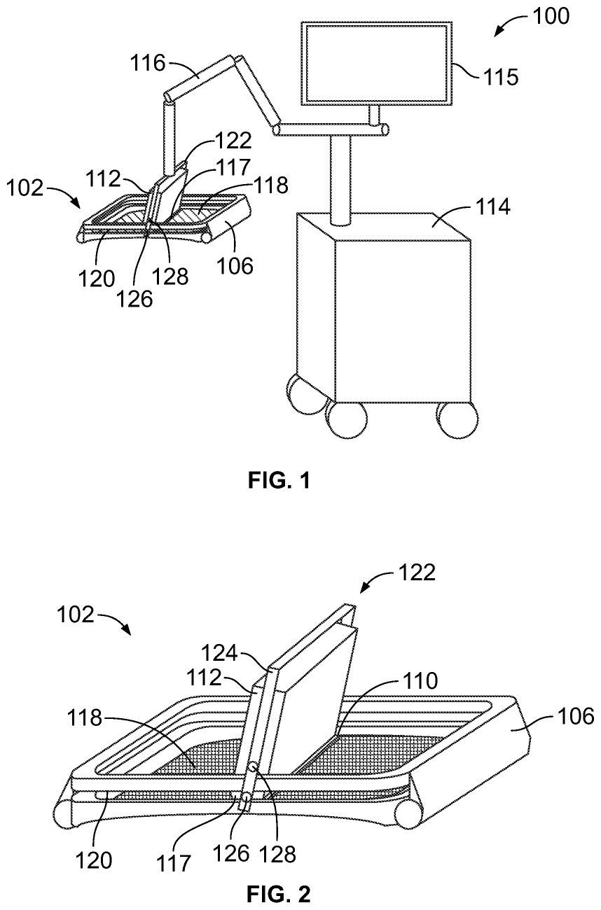

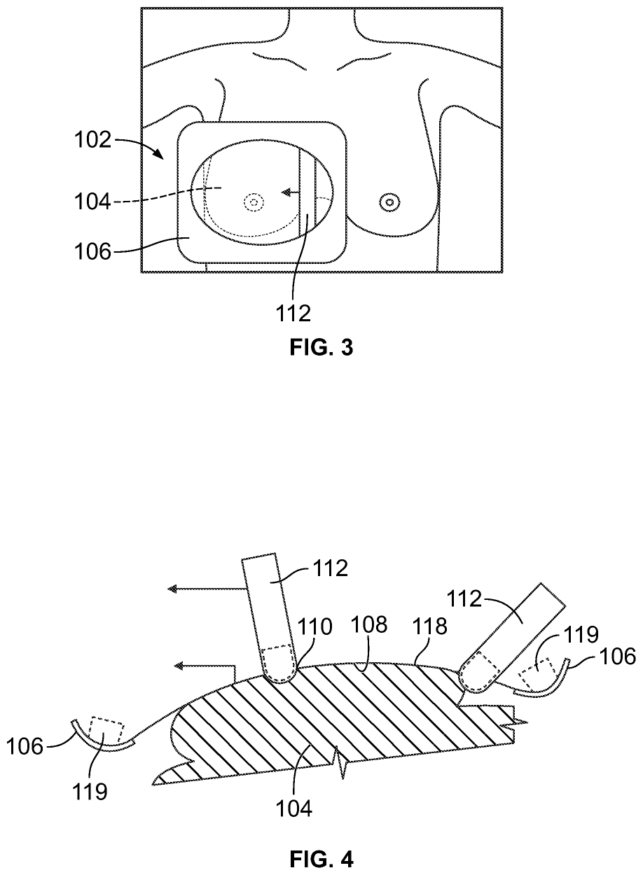

The FIGS. 1-5 illustrate an example imaging system 100. The imaging system 100 includes a compression and scanning assembly 102 for compressing breast tissue 104. The compression and scanning assembly 102 includes a stationary frame 106 with a compression surface 108 that compresses the breast tissue 104. The compression surface 108 includes an open region 110, or aperture, for accommodating an ultrasound transducer 112 such that the ultrasound transducer 112 can directly contact the breast tissue 104 through this open region 110. In particular, the compression surface 108 provides access directly to any region of the breast tissue 104 by forming an open region 110. In one example, the open region can be altered, modified, moved, etc. after compression of the breast tissue 104 occurs by the compression and scanning assembly 102. As a result, the compression and scanning assembly 102 does not have to be removed after attachment to the breast, facilitating testing. In addition, in this manner, the open region 110 is able to be moved, shifted, etc. during scanning.

In one example, the compression surface 108 with the open region 110 may be occupied by an ultrasound transducer 112 that can move over the breast tissue 104 for scanning the breast tissue 104 without the transducer losing contact with the breast tissue. The ultrasound transducer 112 can exert local compression that may be different form the overall compression from the compression surface 108 and may also be different from one location on the breast tissue to another. In particular, a clinician, or automated device may desire to apply a different amount of pressure to the breast tissue 104 at a first breast tissue region than a second breast tissue region. Thus, while the compression and scanning assembly 102 remains static with the same compression on the breast tissue 104, the local pressure of the ultrasound transducer 112 may be varied in the open region 110. Consequently, additional control and functionality is realized. In addition, by making measurements that avoid any hindrance to access, such as from a taut mesh, to areas such as the medial and lateral edges of the breast tissue 104 or the areolar area, improved scanning is provided. In certain embodiments the transducer 112 may have a curved or semi curved profile to match the shape of the breast and allow better access and coverage of areas such as the medial and lateral edges of the breast tissue 104. Alternatively, a group of two or more transducers can be used by holding them together in a manner that better approximates a curved surface profile.

In one example, the movement, compression force, and orientation of the ultrasound transducer 112 can be manual, user assisted, semi-automated, automated, or the like. In yet another embodiment, the ultrasound imaging may be done with the patient lying in a supine position as is done during manual examination, in a prone position, in a standing position, in a sitting position, or in a supine or prone position with the breast tissue 104 compressed against the chest for obtaining ultrasound images in the coronal plane of view. The position of the ultrasound transducer 112 can be tracked by a vision system or a system incorporated into the frame 106 of the compression surface 108 capable of tracking of the location of the open region 110 and/or the ultrasound transducer 112. To this end, the movement, compression force, and orientation of the ultrasound transducer 112 can be manipulated using mechanical or robotic means based on real time feedback from the image. Such feedback may include details of features that require a change in compression force or orientation of the transducer 112 to better visualize the features or to provide a different view of those features. Alternatively, the output from an artificial intelligence (AI) algorithm that analyses the image in real time may be used as feedback.

The imaging system may also include a workstation 114 that may contain one or more processors, beamforming electronics, and other processors and electronics including user interface devices and processors, data management and control, a memory that contains instructions that may be implemented by the one or more processors, power supplies, or the like. The processors of the workstation 114 are configured to receive ultrasound signals and convert the ultrasound signals into ultrasound images. The workstation 114 may include a display 115 for viewing images, inputting information, obtaining data and information, or the like.

An articulating support arm 116 may couple the workstation 114 to the compression and scanning assembly 102. In one example, the articulating support arm 116 autonomously moves to place the compression and scanning assembly 102 at a desired location. Alternatively, the articulating support arm 116 moves as a result of manual operation by a clinician. In particular, for ease of use, the articulating support arm 116 can be equipped with an assist feature that allows the user to freely move and place the compression and scanning assembly 102 on the breast tissue 104 and lock it into place where a positive compression pressure is applied via the compression surface on the breast tissue. The articulating support arm 116, in one example, may also be equipped with the capability to robotically control the movement of the ultrasound transducer 112 including the ability to modulate local pressure with the ultrasound transducer 112 and adjust the orientation of the transducer with respect to the breast tissue surface. The local pressure may be different from the overall or global pressure applied by the compression surface. Alternatively, the frame 106 of the compression and scanning assembly is equipped with the capability to robotically control the movement, orientation, and local pressure of the ultrasound transducer. The support arm and/or the compression and scanning assembly are also equipped to control and modulate the scan speed of the transducer to accommodate the time needed for image processing to obtain the information that will guide the movement, orientation, and local pressure of the ultrasound transducer. The image processing can be accomplished using computer algorithms or visually by an expert clinician.

As best illustrated in FIG. 2, the compression and scanning assembly 102 includes the frame 106 that receives a compression element 118. The compression element 118 in one example is a membrane that is a polymer mesh material, and includes an arcuate surface that is received by the frame 106. The compression and scanning assembly 102 supports the compression surface 108 such that the membrane can be disposed across a bottom opening thereof that compresses the breast tissue 104, usually toward the rib cage.

In one embodiment, the compression element 118 may be an acoustically transparent (sonolucent) polymer membrane that serves the dual purpose of coupling ultrasound energy from the transducer into the tissue of the breast tissue 104 as well as providing a lubricating surface to the moving elements of the compression surface 108 and to the ultrasound transducer 112. In one example, the membrane can be made of a silicone hydrogel, poly hydroxyethyl methacrylate (pHEMA) or other similar material. The compression element 118 can be placed on the breast tissue 104 prior to compression to help streamline the procedure. Alternatively, the compression surface 108 has gaps or holes that do not substantially compromise compression while allowing application of ultrasound gel onto the skin surface prior to performing an ultrasound scan of the breast tissue. Ultrasound gel is used to couple the acoustic energy from the ultrasound transducer into the tissue without crossing through air at any point. The presence of the ultrasound gel has the effect of acting as a lubricant permitting movement of the surface of the compression surface 108 and ultrasound transducer over the breast tissue surface with minimal patient discomfort.

In one example, sensors 119 are placed within the frame 106 on the compression element 118 to detect the characteristics of the compression element 118. In one example the sensors 119 are pressure sensors that in one example are piezo-electric type pressure sensors, although other types of sensors could be used including strain gauges and/or MEMs bases pressure sensors. The pressure sensors on the frame 106 of the compression and scanning assembly measures the global pressure being applied to the patient's breast tissue 104. Optionally, a pressure sensor may also be integrated in the ultrasound transducer to measure the local pressure. Both global and local pressure aid in scanning consistency and can be connected to a feedback control loop to provide a more constant force to the patient's breast tissue 104, thereby further increasing the consistency of scans.

The frame 106 also includes a slot 120 that receives a rock and tilt assembly 122. In one example the slot 120 continuously extends about the perimeter of the frame 106. Optionally, the slot 120 comprises multiple slots disposed about the perimeter of the frame. In particular, the rock and tilt assembly 122 includes a subframe 124 that receives the ultrasound transducer 112, and is coupled to the frame with a lower pin element 126 that is disposed through the slot 120 and an upper pin element 128 that engages the top surface of the frame spaced from the slot. In this manner the lower pin element 126 and upper pin element 128 can slide forwards and backwards, while also providing two spaced pivot points coupling to the frame. As a result, the orientation of the ultrasound transducer 112 can be tilted, rocked, or twisted to direct ultrasound energy into the breast tissue at different angles and for maintaining adequate contact with the breast tissue 104. For example, when near the medial or lateral edge of the breast tissue the transducer may be tilted to direct ultrasound energy into the breast tissue 104.

In one example, the ultrasound transducer 112 inside this assembly 102 occupies the open region 110 in the compression surface 108 and is swept over the compressed breast tissue 104 along with the compression surface 108 in a generally arcuate trajectory to ultrasonically scan the compressed breast tissue 104. The open region 110 allows direct contact between the ultrasound transducer 112 and the breast tissue 104, and is equipped with an engagement element 117 to engage and release with complimentary features on the ultrasound transducer 112. The compression surface 108 in one example is translucent or made of elements that are translucent to visible light to allow the user to see the breast tissue therethrough for facilitating ease of positioning.

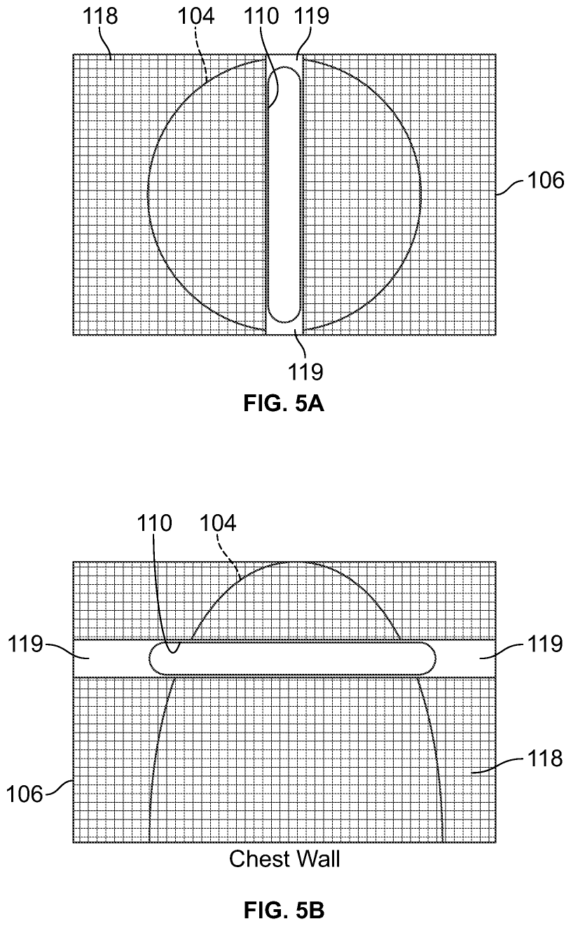

FIGS. 5A and 5B are schematic diagrams of a breast tissue surface in accordance with aspects of the present disclosure. FIG. 5A depicts the compression surface used in the ABUS (automated breast ultrasound) configuration where the patient is lying supine or prone with the breast tissue positioned to be scanned for capturing coronal views. The 3D volumetric data recorded in the ultrasound measurement can then be used to reconstruct 2D images as needed. For example 2D coronal slices of the breast tissue can be generated for evaluation.

FIG. 5B depicts the compression surface used in multimodality mammography and ultrasound imaging. In this case the compression and scanning assembly which includes the compression surface replaces the standard compression surface (also known as a paddle) used in mammography. Here ultrasound scanning is done with the breast tissue positioned for capturing CC or MLO views i.e., similar to views captured in mammography. The 3D volumetric data recorded in the ultrasound measurement can then be used to reconstruct 2D images as needed. For example 2D transverse slices of the breast tissue similar to those obtained in x-ray mammography can be generated for evaluation.

As illustrated, the compression surface 108 has an open region 110 or aperture in which an ultrasound transducer 112 is placed for direct contact with the breast tissue 104. This open region 110 does not interfere with the mammography and can also be moved out of the way over to one side of the compression surface during mammography. During ultrasound scanning the open region 110 is occupied by a transducer 112 that exerts compression pressure that can be varied as needed. The position of the open region can be tracked using sensors 119 on the paddle frame or sensors on an external arm 116 controlling the movement of the transducer. The open region 110 has one or more compression structures to engage the ultrasound transducer. Similarly, the transducer 112 is equipped with one or more complementary structures configured to compress and release the compression structures on the open region. The movable elements may be completely rigid or allow a certain degree of flexing to better accommodate the breast tissue which is generally conical and curved in shape.

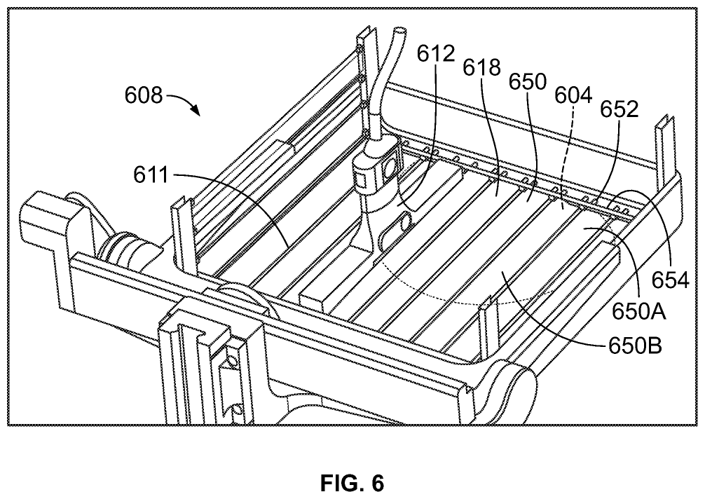

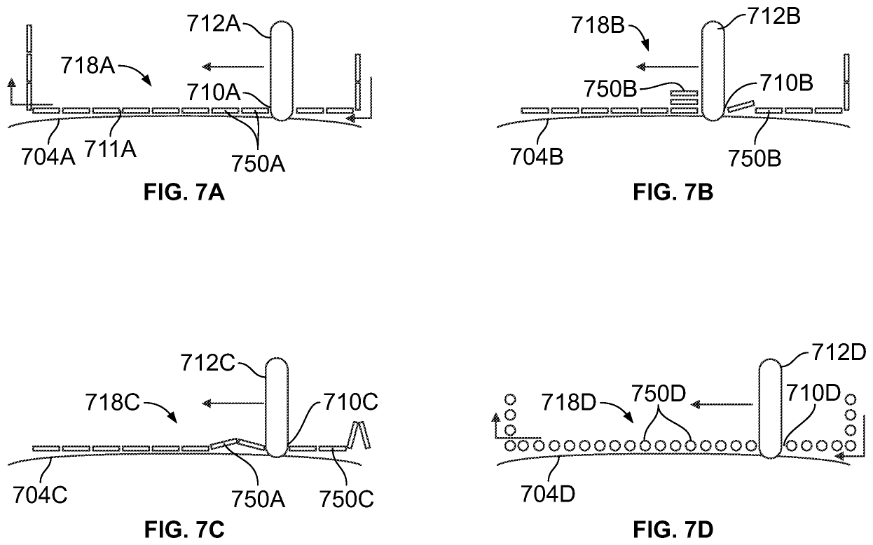

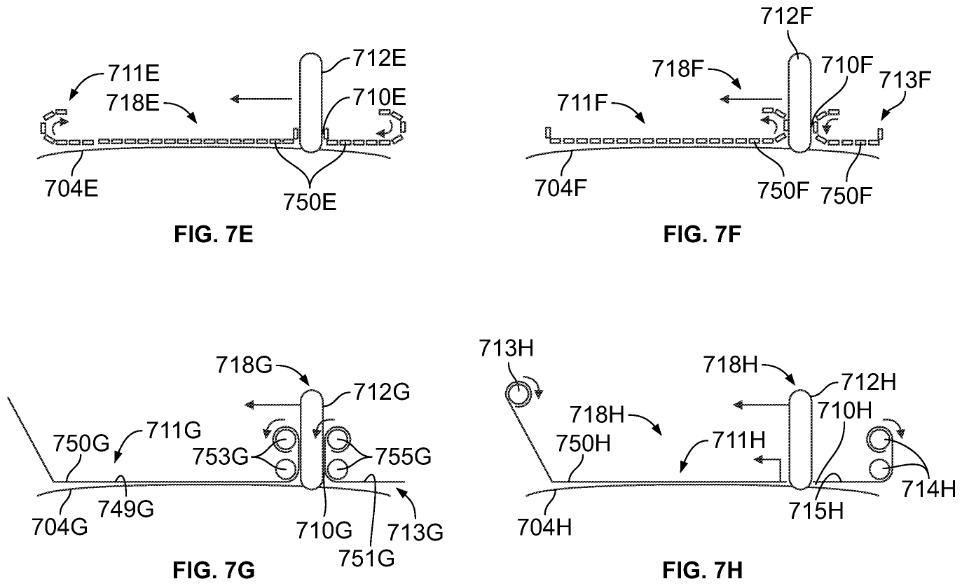

FIGS. 6 and 7A-7H illustrate additional embodiments that utilize a compression paddle 608 that includes numerous alternative compression elements 618 and 718A-H that each provide a region opening 710A-H. Instead, the imaging system 600 includes numerous compression elements 718A-H that compress the breast tissue 604, 704A-H where each compression element 618, 718A-H includes a region opening 710A-H for direct contact of an ultrasound transducer 612, 712A-H to the tissue of the breast tissue 604, 704A-H.

In the embodiment of FIG. 6, the compression paddle 608 includes a compression element 618 that includes plural movable elements 650 that are slidable coupled within the compression paddle 608. The plural movable elements can include a first movable element 650a, a second movable element 650b, etc. where region opening can be disposed within the moveable elements. In one example, each movable element 650 has a prong element 652 that is received within a slot 654 of the compression paddle 608. In one example, each movable element 650 is made from a radio transparent material. In another example, each movable element is spaced apart from an adjacent movable element 650 to form plural channels 611 across the compression element 618.

FIG. 7A illustrates one example of how the movable elements of FIG. 6 may be moved, modified, etc. to form a region opening 710A in a compression element 718A. Similar to FIG. 6, the compression element 718A includes plural movable elements 750A spaced from one another via channels 711A. As illustrated in FIG. 7A, in this embodiment each individual movable element 750A may be moved transversely to form a region opening 710A for the ultrasound transducer 712A. In one example the movable elements 750A may be manually moved by a clinician, ultrasound transducer 712A, etc. In other example embodiments the movable elements may be moved mechanically, include by a motor and gear arrangement, conveyor system, or the like.

FIG. 7B illustrates another example of how the movable elements of FIG. 6 may be moved, modified, etc. to form a region opening 710B in a compression element 718B. In this embodiment, the plural movable elements 750B may be removed and/or stacked on top of one another. Again, the removal, stacking, etc. may be accomplished manually, or through mechanical, electrical, or other automatic manner.

FIG. 7C illustrates yet another example of how the movable elements of FIG. 6 may be moved, modified, etc. to form a region opening 710C in a compression element 718C. In this embodiment, the plural movable elements 750C may be hingedly coupled to one another such that by moving the movable elements 750C transverse to form the region opening 710C, the movable elements accordion together. Therefore, the region opening 710C is formed to accommodate the ultrasound transducer 712C by moving the movable elements manually, or automatically.

FIG. 7D illustrates an example when the movable elements 750D of the compression element 718D are plural side by side roller elements. Each roller element may be slidably received within a slot of the compression element 718D such that compression of the ultrasound transducer 712D against a roller element causes lateral movement of roller elements resulting in the forming of a region opening 710D and allowing the transducer to scan the breast tissue while in direct contact with it.

FIG. 7E illustrates an example embodiment again with a compression element 718E including side by side movable elements 750E. In this example the movable elements form a conveyer 711E that wraps around two gears and includes the region opening 710E. As the movable elements 750E rotate about the gears the region opening 710E similarly moves. In this manner, the ultrasound transducer 712E can move with the region opening 710E. In one example, the ultrasound transducer 712E is held in place by the movable elements, or a securing device, and the conveyor 711E is autonomously moved to move the ultrasound transducer along the tissue.

FIG. 7F illustrates another example embodiment with a compression element 718F including side by side elements 750F. In this arrangement, two separate sections of elements 750F are provided with the region opening 710F disposed between a first section 711F and the second section 713F. Rolling up of the first section 711F and unrolling of the second section 713F result in movement of the region opening 710F. Similar to the embodiment of FIG. 7E, the first and second conveyors 711F and 713F may be moved autonomously.

FIG. 7G illustrates yet another example embodiment of a compression element 718G. In this example embodiment, a first membrane section 749G and second membrane section 751G are provided that are rolled up ahead of the movement of the transducer by the moveable elements 750G. The moveable elements, in this example, include first rollers 753G and rolled off a second rollers 755G respectfully. In this example, the first rollers 753G and second rollers 755G are dynamically move side to side in order to move the region opening 710G. The membrane having the first membrane section 749G second membrane section 751G in one example is any membrane as described in relation to FIGS. 1-5. The region opening 710G is disposed between the first and second membrane sections is occupied by the ultrasound transducer 712G and may be moved manually or automatically as a result of rotation of the first and second rollers 753G and 755G. In addition, in one example, the compression element extends at an angle over the patient's chest and towards her neck in order to better access the tissue near the chest wall.

FIG. 7H illustrates another example embodiment of a compression element 718H. In this example embodiment, similar to the embodiment of FIG. 7G, a membrane 750H is utilized as compression surface with a first membrane section 711H and roller 713H that rolls up the membrane ahead of the movement of the transducer. Meanwhile a second membrane section 715H is rolled off rollers 714H behind the movement of the transducer. Unlike FIG. 7G the rollers 713H and 714H remain static such that the membrane in 7H moves relative to the breast surface due to the action of the rollers and movement of the ultrasound transducer 712H. In addition, in one example, similar to the embodiment of FIG. 7G, the compression element extends at an angle over the patient's chest and towards her neck in order to better access the tissue near the chest wall.

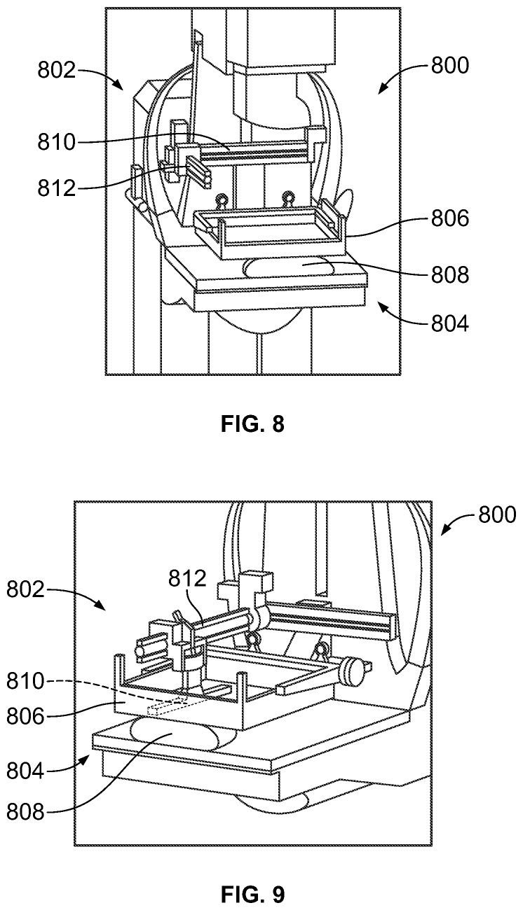

FIGS. 8 and 9 illustrate yet another embodiment where the ultrasound imaging may be done in tandem with x-ray imaging to obtain images in both modalities in the same patient encounter and in approximately the same time. The x-ray imaging and ultrasound imaging may be conducted with the patient while standing as is commonly done or while sitting or while lying in a decubitus position. In an alternate embodiment the patient may be positioned prone such as on a biopsy table with the breast tissue hanging pendant through a hole in the table. One or more mammography or tomosynthesis images are acquired of the breast tissue in the CC or MLO (cranio-caudal or medio-lateral-oblique) or beyond positions i.e. in the approximately transverse or sagittal positions followed by scanning with the ultrasound transducer to obtain images in the same position.

The compression pressure may be reduced immediately following x-ray mammography and before commencing ultrasound scanning. While compression is necessary for obtaining images in the same configuration as mammography, ultrasound imaging may not require the level of thickness reduction or thickness uniformity as mammography. This is because unlike x-ray mammography which uses a single exposure setting (x-ray tube power and detector gain) when capturing and image of the entire breast tissue and where the thickness needs to be made as small as can be tolerated by the patient to reduce x-ray dose ultrasound is a scanning approach and the sound frequency and dwell times can be changed according to tissue thickness. The compression pressure can therefore be relaxed to a more tolerable level for ultrasound scanning. This is important because while x-ray image acquisition takes only a few seconds, ultrasound scanning can take a longer time, such as up to a minute, making it difficult for the patient to tolerate the vigorous compression used in mammography for such a long time. Indeed, given that the human breast tissue is somewhat cone shaped with the posterior or portion at the chest being larger than the anterior, the vigorous flat compression used in mammography causes the posterior to be compressed too much causing pain and discomfort while the anterior part of the breast tissue may not be compressed at all. Therefore, for the ultrasound measurement the compression may be tapered or curved to make it more tolerable for the patient. Nonetheless, a 2D (two dimensional) ultrasound image can be reconstructed from the 3D volumetric ultrasound image data which is sufficiently representative of the x-ray mammography image for side by side image evaluation and comparison for better detection of breast tissue abnormalities.

As illustrated, the imaging system 800 of FIG. 8 includes an ultrasound breast tissue imaging assembly 802 mechanically coupled to an x-ray imaging system 804. In one example, the ultrasound breast tissue assembly is the imaging system of FIGS. 1-5. In other embodiments, the compression element 806 of the ultrasound breast tissue imaging assembly may be any of the compression elements described in relation to FIGS. 1-7H. In addition, the compression element of the ultrasound breast tissue imaging assembly 802 is also a compression element of the x-ray imaging system. As illustrated, in the example embodiment of FIGS. 8 and 9 the compression element 806 is a paddle that compresses the breast tissue 808 and compresses the breast tissue 808 between the compression element 806 and an x-ray detector 808. In one example, an ultrasound transducer 810 is placed on an arm element 812 that is moveable from a first position when ultrasound scanning is occurring, to a second position away from the compression element 806 when x-rays are being obtained. In one example, the arm element 812 is autonomously moveable from the first position to the second position. In this manner, the imaging system 800 is able to capture x-ray images and ultrasound images during one visit to a clinician, improving efficiencies and increasing the amount of data gathered during a visit.

In an alternative embodiment a standard breast mammography compression paddle can be switched out with an ultrasound enabling compression paddle such as the shown in FIG. 8 immediately following a mammography imaging procedure. The patient's breast is then scanned as described above with an ultrasound transducer to generate 3D volumetric ultrasound image data from which a 2D (two dimensional) ultrasound image can be reconstructed that is sufficiently representative of the x-ray mammography image for side by side image evaluation and comparison for better detection of breast tissue abnormalities

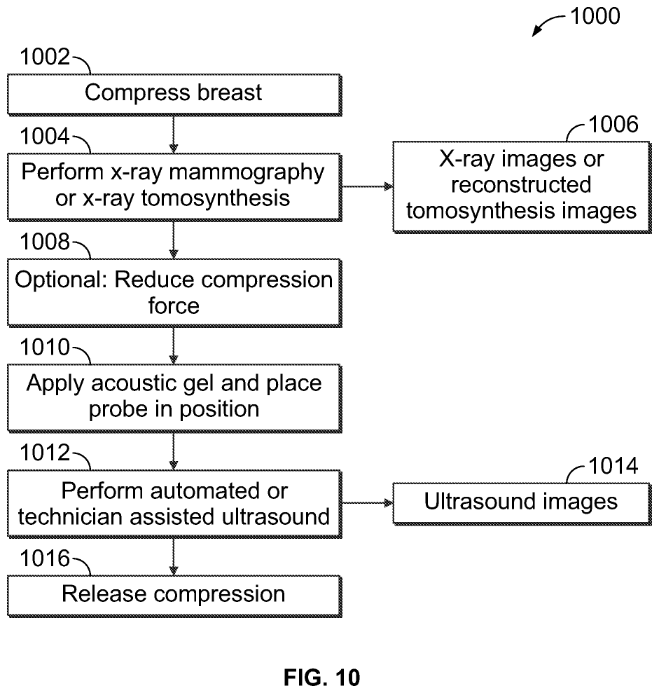

FIG. 10 illustrates yet another example embodiment of an imaging system 1000. In this example embodiment, the imaging system 1000 includes a compression and scanning assembly 1002 for compressing breast tissue. The compression and scanning assembly 1002 includes a frame 1006 with a first compression element 1007 that includes compression surface 1008 that compresses the breast tissue against a second compression element 1009. In one example, the first compression element 1007 is a breast paddle that is moveable in relation to the second compression element 1009. In one example, the second compression element 1009 is moveable and placed in a stationary position such that the breast may be placed on the second compression element 1009, and then the first compression element 1007 is moved relative to the second compression element 1009 to compress the breast tissue between the first compression element 1007 and second compression element 1009.

In an example, the first compression element 1007 includes an open region 1010, or aperture, for accommodating an ultrasound transducer 1012 such that the ultrasound transducer 1012 can directly contact the breast tissue through this open region 1010. The first compression element 1007 in example embodiments may be any of the compression elements discussed in relation to FIGS. 1-7H. In an example, the open region 1010 provides access directly to any region of the breast tissue. In one example, the open region 1010 can be altered, modified, moved, etc. after compression of the breast tissue occurs by the compression and scanning assembly 1002 as described in other embodiments and examples herein. As a result, the compression and scanning assembly 1002 does not have to be removed after attachment to the breast, facilitating testing. In addition, in this manner, the open region 1010 is able to be moved, shifted, etc. during scanning. In particular, a clinician, or automated device may desire to apply a different amount of pressure to the breast tissue at a first breast tissue region than a second breast tissue region. Thus, while the compression and scanning assembly 1002 remains static with the same compression on the breast tissue, the local pressure of the ultrasound transducer 1012 may be varied in the open region 1010. Consequently, additional control and functionality is realized.

In one example, the movement, compression force, and orientation of the ultrasound transducer 1012 can be manual, user assisted, semi-automated, automated, or the like. In yet another embodiment, the ultrasound imaging may be done with the patient lying in a supine position as is done during manual examination, in a prone position, in a standing position, in a sitting position, or in a supine or prone position with the breast tissue compressed against the chest for obtaining ultrasound images in the coronal plane of view. The position of the ultrasound transducer 1012 can be tracked by a vision system or a system incorporated into the frame 1006 of the compression surface 1008 capable of tracking of the location of the open region 1010 and/or the ultrasound transducer 1012. To this end, the movement, compression force, and orientation of the ultrasound transducer 1012 can be manipulated using mechanical or robotic means based on real time feedback from the image. Such feedback may include details of features that require a change in compression force or orientation of the transducer 1012 to better visualize the features or to provide a different view of those features. Alternatively, the output from an artificial intelligence (AI) algorithm that analyses the image in real time may be used as feedback.

The imaging system 1000 may also include a workstation 1014 that may contain one or more processors, beamforming electronics, and other processors and electronics including user interface devices and processors, data management and control, a memory that contains instructions that may be implemented by the one or more processors, power supplies, or the like. The processors of the workstation 1014 are configured to receive ultrasound signals and convert the ultrasound signals into ultrasound images. The workstation 1014 may include a display 1015 for viewing images, inputting information, obtaining data and information, or the like.

An articulating support arm 1016 may couple the workstation 1014 to the compression and scanning assembly 1002. In one example, the articulating support arm 1016 autonomously moves to place the compression and scanning assembly 1002 at a desired location. Alternatively, the articulating support arm 1016 moves as a result of manual operation by a clinician. In particular, for ease of use, the articulating support arm 1016 can be equipped with an assist feature that allows the user to freely move and place the compression and scanning assembly 1002 on the breast tissue and lock it into place where a positive compression pressure is applied via the compression surface on the breast tissue. The articulating support arm 1016, in one example, may also be equipped with the capability to robotically control the movement of the ultrasound transducer 1012 including the ability to modulate local pressure with the ultrasound transducer 1012 and adjust the orientation of the transducer with respect to the breast tissue surface. The local pressure may be different from the overall or global pressure applied by the compression surface. Alternatively, the frame 1006 of the compression and scanning assembly is equipped with the capability to robotically control the movement, orientation, and local pressure of the ultrasound transducer. The support arm and/or the compression and scanning assembly are also equipped to control and modulate the scan speed of the transducer to accommodate the time needed for image processing to obtain the information that will guide the movement, orientation, and local pressure of the ultrasound transducer. The image processing can be accomplished using computer algorithms or visually by an expert clinician.

In an additional embodiment as illustrated in FIG. 11, a method for acquiring breast tissue data 1100 is provided. In one embodiment, an imaging system as described in any of the previous Figures may be utilized to implement the process. During the process 1100, breast tissue is compressed with the help of a compression assembly described above and placed into the field of view and maintained in this position. One or more x-ray mammography images are acquired of the breast tissue. The open region can be moved over to a side of the compression surface during the x-ray imaging (to be out of the way of the x-ray beam) and is brought to a position on the breast tissue for the ultrasound imaging and an ultrasound transducer is positioned on the breast tissue through the open region. In certain embodiments, the ultrasound transducer is at least 15 cm long (such as 19 cm to 30 cm long) to span the entire breast tissue in one pass of the transducer. Alternatively, in another embodiment, two or more ultrasound transducers may be lined up (linearly arranged) so as to form a longer transducer length. An advantage of this is that the angle between the transducers may be altered to better contact the curved breast surface. In addition, in certain embodiments, the ultrasound transducer may have surfaces curved to better match the curvature of the breast. In addition, as discussed herein, the ultrasound transducer may be configured for a fast readout, such that the ultrasound scan can be performed in a minute or less.

At 1102, the breast tissue is initially compressed to the desired thickness as required for x-ray imaging. At 1104, the x-ray imaging is provided. The x-ray imaging in one example takes anywhere from about one second to few seconds to complete depending on whether it is a single image as in standard mammography or multiple images taken for tomosynthesis. At 1106, the images may be transferred for further processing and reconstruction as needed.

At 1108, optionally, the compression can be released and or modified. In particular, unlike conventional radiographic mammography approaches, the compression does not need to result in a uniform thickness of tissue being imaged and may instead be more accommodative of the breast shape by allowing a tapered or curved compression. In one example, different compression paddle may optionally be used replacing the paddle used in mammography. In one example, the compression is performed using a rigid or semi-rigid paddle structure that is comprised of movable elements designed to move over the breast tissue surface along with an ultrasound transducer that is placed in direct contact with the breast tissue. The movable elements along with the transducer apply the necessary compression force on the breast tissue except that the local compression force applied by the ultrasound transducer can be different from that of the overall compression of the paddle. In certain embodiments, feedback on compression force measured by sensors or other indicators may be used to determine when sufficient compression is achieved. stopped, i.e., when sufficient contact is established. For example, in one embodiment, compression of the breast tissue may be based on a threshold criterion. In another embodiment compression may be determined based on feedback from the patient or the technician.

At 1110, ultrasound gel may be applied on the breast tissue. In one example, the gel is applied prior to applying compression. Alternatively, the gel is applied through channels between the movable elements, such as movable elements, or apertures within the moveable elements.

At 1112, the ultrasound scan is initiated by first placing the transducer on the breast tissue surface through an open region between the movable elements. Once positioned, the ultrasound transducer may be mechanically or electromechanically driven along a defined path and may, while driven, acquire ultrasound image data of the underlying breast tissue. In certain embodiments a technician holds the transducer and controls the transducer compression and tilt while he or she scans the breast tissue. Alternatively, the compression and tilt are assisted by a technician who can alter the local compression and tilt of the transducer during automated movement of the transducer.

The ultrasound scan of the breast tissue can be conducted in one or more passes over the breast tissue surface. In one embodiment the scan may be conducted in a medial-lateral direction while in another embodiment it may be conducted in an anterior-posterior direction. In certain embodiments, an automated ultrasound scanning system may be used to implement a pre-programmed scan protocol in an automated manner. Such scans may be performed by moving an ultrasound transducer across the breast tissue of a patient without user intervention or guidance during the scan operation. The position of the ultrasound transducer can be recorded based on its position within the rigid paddle frame using position sensors or using an external vision system.

At 1114, the captured images are then used to reconstruct and render 3D ultrasound images of the breast tissue. These images can then be used for comparison with the x-ray images and for evaluation and review. At 1116, following ultrasound imaging the compression is released.

FIG. 12 illustrates a process for acquiring breast tissue data 1200. In one embodiment, an imaging system as described in any of the previous Figures may be utilized to implement the process. During the process 1200, breast tissue is compressed with the help of a compression assembly described above and placed into the field of view and maintained in this position until signals from an ultrasound transducer are obtained and analyzed. Based on the analysis of the signals from the ultrasound transducer, the ultrasound transducer is moved to reposition the ultrasound transducer to facilitate imaging.

At 1202, breast tissue is initially compressed to a determined thickness. The determined thickness may be determined based on the size of the breast, area being examined, historical data related to other patients, historical data related to the patient, a setting value determined prior the examination, a set value manually input by a clinician prior to examination, or the like. The initial determined thickness may be determined by a clinician, artificial intelligence algorithm, etc.

At 1204, one or more processors obtain signals from the ultrasound transducer at the initial position, and determined thickness. In particular, the signals generated by the ultrasound transducers are received for analysis.

At 1206, the one or more processors analyze the signals obtained from the ultrasound transducers. In one example, an artificial intelligence algorithm is utilized by the one or more processors to determine if a better compression, position of the ultrasound transducer, etc. can be provided to receive better signals, additional data, or the like during a scan. While in one example this process may be fully automated including use of the artificial intelligence algorithm, in another example, the analysis may be semi-automated, performed by a clinician, etc.

At 1208, the one or more processors determine if movement of the ultrasound transducer, or compression adjustment, is needed. If not, at 1210, scanning continues. If movement or adjustment is required, then at 1212, the one or more processors may adjust the compression of an engagement member, or reposition the ultrasound transducer. In one example, to adjust compression, a paddle compression element may be adjusted. In another example, the ultrasound transducer may be coupled to and move by an articulating arm that repositions the ultrasound transducer. After movement to the second position, the process may repeat until a determination is made that enough imaging data has been obtained.

### CLOSING STATEMENTS

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices, and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A imaging system comprising:
a compression and scanning assembly including a frame that receives a compression element configured to compress breast tissue, the compression element including an open region configured to receive an ultrasound transducer;
wherein the open region is configured to move relative to the breast tissue after the compression and scanning assembly has been secured to the breast tissue.

2. The imaging system of claim 1, further comprising:
an articulating support arm coupled to the compression and scanning assembly and configured to permit movement of the compression and scanning assembly.

3. The imaging system of claim 2, further comprising a work station coupled to the articulating arm, and including one or more processors configured to:
move the articulating support arm,
operate the ultrasound transducer to move the ultrasound transducer relative to the compression and scanning assembly.

4. The imaging system of claim 1, further comprising a work station including one or more processors configured to:
obtain signals from the ultrasound transducer;
analyze the signals from the ultrasound transducer utilizing an artificial intelligence algorithm; and
automatically moving the ultrasound transducer based on the analysis of the signals.

5. The imaging system of claim 1, wherein the compression element is a membrane formed from a polymer mesh material.

6. The imaging system of claim 1, wherein the compression element includes at least one movable element configured to actuate to move the open region from a first position to a second position.

7. The imaging system of claim 1, wherein the compression element is a compression paddle that includes a first movable element spaced from a second movable element to form a channel therebetween.

8. The imaging system of claim 7, wherein the first movable element is removable from the compression paddle.

9. The imaging system of claim 7, wherein the first movable element is hingedly coupled to the second moveable element.

10. The imaging system of claim 1, wherein the compression element includes at least one movable element that is a roller element.

11. The imaging system of claim 1, further comprising, an x-ray detector coupled to the compression and scanning assembly.

12. The imaging system of claim 10, further comprising a movable arm configured to receive the ultrasound transducer and to move the ultrasound transducer away from the x-ray detector or x-ray mammography imaging area.

13. The imaging system of claim 1, wherein the compression and scanning assembly further comprises a rock and tilt assembly received within the frame and configured to receive the ultrasound transducer; wherein the rock and tilt assembly includes a subframe that is configured to receive the ultrasound transducer and is slidably coupled to the frame with a lower pin element and an upper pin element, wherein the subframe is configured to rotate the ultrasound transducer about the lower pin element and upper pin element.

14. A method for imaging comprising:
compressing breast tissue to a first thickness with a compression element of a compression and scanning assembly;
x-ray imaging the breast tissue at the first thickness with the compression and scanning assembly to obtain an x-ray image;
disposing an ultrasound traducer through an open region in the compression element to directly compress the breast tissue; and
moving the ultrasound transducer across the breast tissue as the open region is modified in the compression element to obtain an ultrasound image.

15. The method of claim 14, further comprising:
applying gel to the breast tissue through channels in the compression element of the compression and scanning assembly prior to disposing the ultrasound transducer through the open region in the compression element.

16. The method of claim 14, wherein x-ray imaging the breast tissue at the first thickness with the compression and scanning assembly to obtain an x-ray image includes reconstructing an initial x-ray image to form the x-ray image.

17. The method of claim 14, further comprising, modifying compression of the breast tissue with the compression and scanning assembly after x-ray imaging the breast tissue and before disposing the ultrasound transducer through the open region of the compression element.

18. The method of claim 17, wherein modifying the compression of the breast tissue includes obtaining feedback related to a compression force measured by at least one sensor, and modifying the compression based on the feedback obtained.

19. A imaging system comprising:
a compression and scanning assembly including a frame that receives a compression element configured to compress breast tissue, the compression element including moving elements with an open region formed within the moving elements, the moving elements configured to receive an ultrasound transducer within the open region;
wherein a position of the open region in the moving elements is configured to move relative to the breast tissue after the compression and scanning assembly has been secured to the breast tissue;
an x-ray detector coupled to the compression and scanning assembly for obtaining x-ray images.

20. The imaging system of claim 19 further comprising a support arm coupled to the compression and scanning assembly and configured to move the compression and scanning assembly away from the x-ray detector when the x-ray detector is obtaining the x-ray images.
